(19) United States
(12) Patent Application Publication
(10) Pub. No.: US 2006/0129221 A1
(43) Pub. Date: Jun. 15, 2006

(54) Tunneling guide

(75) Inventor: Kenneth T. Heruth, Edina, MN (US)

Correspondence Address: MEDTRONIC, INC., 710 MEDTRONIC PARK, MINNEAPOLIS, MN, 55432-9924, US

(73) Assignee: Medtronic, Inc., Minneapolis, MN (US)

(21) Appl. No.: 11/069,187

(22) Filed: Mar. 1, 2005

Related U.S. Application Data
(60) Provisional application No. 60/635,407, filed on Dec. 10, 2004.

(51) Int. Cl. A61N 1/05

(52) U.S. Cl. 607/120

(57) ABSTRACT

An implantable tunneling guide capable of releasing one or more therapeutic agents is described. The tunneling guide has a body member defining a lumen. The lumen is configured to receive at least a portion of a therapy delivery element, such as a catheter, a lead, or a lead extension. One or more therapeutic agents are disposed on, in, or about the body member.

### FIELD

The present disclosure relates to generally to implantable medical devices.

### BACKGROUND

Implantation of medical devices, such as pacemakers, neurostimulators, implanted drug pumps, leads, catheters, etc, has been associated with adverse consequences, such as formation of scar tissue surrounding the implant, infection due to bacteria introduced during implantation, and tissue proliferation in blood vessels after a stent implantation. Attempts to prevent or control such adverse reactions have included administration of drugs, completely separate from the intended primary therapy of the implanted medical device. In some cases, systemically administered drugs, e.g. orally, intravenously, or intramuscularly administered drugs, have proven effective in treating complications due to medical device implantation. In other cases, systemic delivery has been ineffective due to, e.g., pharmacokinetic or pharmacodynamic characteristics of the drug, the location of the implanted device, or side effects of the drug. To increase effectiveness in these situations, some implanted devices have been modified to elute the drug into the surrounding tissues.

One common way of providing local drug elution is to dispose a polymer layer on the implantable medical device and embed the drug into the polymer during manufacturing. When hydrated after implant, the drug diffuses out of the polymer into surrounding tissue. Various methods of impregnating polymers with drugs have been used, including mixing the drug into the melted polymer prior to processing (e.g. molding or extrusion), and diffusing the drug into a finished polymer component using chemicals to swell the polymer for rapid loading. In some cases, the implantable medical device (IMD) is made from a polymer that is compatible with the drug, and the drug can be loaded directly into the device. However, incorporation of a therapeutic agent into or onto polymeric material compromises the structural integrity of the material.

Structural integrity of catheters and leads, especially those intended to be chronically or permanently implanted, are important. Such catheters and leads, which are typically made using standard polymeric tubing, such as silicone or polyurethane, are tunneled subcutaneously from a pocket into which an active device, such as a drug pump or neurostimulator, is implanted to the therapy delivery site. For neurological systems, the therapy delivery site is typically the spinal intrathecal space, the spinal epidural space, the ventricles of the brain, or brain parenchyma. For cardiac systems, the therapy delivery site is the heart. Because, the catheters and leads are implanted long term and may be tunneled through a subcutaneous path, it is not desirable to compromise the structural integrity of such devices.

In addition, the active agent disposed on or in the structural body of a catheter or lead can diffuse inward to the catheter drug path or the lead conductors as readily as it diffuses outward to the subcutaneous tissue. For a catheter, it is desirable to completely separate the drug pathway from the active agent. The active agent is intended to be delivered to subcutaneous tissue, and may have deleterious effects at the site of therapy delivery, e.g. the central nervous system (CNS). For stimulation leads, the active agent could cause corrosion of the metallic conductors or electrodes. For both devices, adding the active agent is an extra manufacturing process that complicates manufacturing and may increase scrap.

### BRIEF SUMMARY

In various embodiments, the invention provides a tunneling guide into, onto, or about which a therapeutic agent is disposed rather than associating such an agent directly with a therapy delivery element, such as a catheter or lead. In practice, the tunneling guide may be tunneled through subcutaneous tissue, and then a therapy delivery element may be simply passed through the tunneling guide.

Various embodiments of the embodiment provide an implantable tunneling guide comprising a body member defining a lumen and one or more therapeutic agents disposed on, in, or about the body member. The lumen is configured to receive at least a portion of a therapy delivery element. The lumen may comprise a diffusion barrier to prevent at least a percentage of at least one of the one or more therapeutic agents from diffusing through the lumen. The body member of the tunneling guide may comprise a coating layer and the one or more therapeutic agents are disposed on, in, or about the coating layer.

Various embodiments of the invention provide a system comprising a therapy delivery element and an implantable tunneling guide. The tunneling guide comprises a body member defining a lumen and one or more therapeutic agents disposed on, in, or about the body member. The lumen is configured to receive at least a portion of the therapy delivery element. The system may further comprise a barrier layer disposed between the body member of the tunneling guide and the therapy delivery element. The lumen of the tunneling guide comprises the barrier layer. Alternatively, the barrier layer may comprise a tube separate from the tunneling guide and the therapy delivery element, or the barrier layer may be disposed on the therapy delivery element.

Various embodiments of the invention provide a method for delivering a therapeutic agent associated with implantation of a therapy delivery element. The method comprises implanting a tunneling guide within a patient and inserting at least a portion of the therapy delivery element into the lumen of the tunneling guide. The tunneling guide comprises a lumen defined by a body member and one or more therapeutic agents disposed on, in, or about the body member. The method may further comprise lubricating the lumen to facilitate insertion of the at least a portion of the therapy delivery element into the lumen. The method may further comprise disposing a barrier layer between the body member of the tunneling guide and the therapy delivery element.

One or more embodiments of the present invention may provide advantages over existing technology. For example, separation of a therapeutic element from a therapy delivery element, such as a catheter or a lead, may result in increased structural integrity of the catheter or lead. Additionally, the addition of a tunneling guide or barrier layer in close proximity to an outer diameter of a catheter or lead may result in increased mechanical strength of the catheter or lead. Further, since the tunnel guide is simply a length of tubing rather than a complex assembly, manufacturing is a much simpler process. The active agent is separated from the catheter or lead, and only a small amount can diffuse into the catheter or lead. An additional benefit of this design is that the catheter or lead will be easier to remove and replace if necessary. The body's natural defense mechanisms encapsulate any foreign body that is implanted. This encapsulation, or scar tissue, may also attach to the foreign body. If a catheter or lead has to be removed or replaced for any reason, it can be difficult to remove from the scar tissue. When using the tunnel guide, the scar tissue will encapsulate and attach to the tunnel guide, and leave a clear pathway through the lumen of the tunnel guide. The existing catheter or lead can simply be pulled out and the new device can be inserted through the intact tunnel guide. These and other advantages will become evident upon reading the disclosure presented herein.

### BRIEF DESCRIPTION OF THE DRAWINGS

FIGS. 1A-1B are diagrammatic illustrations of longitudinal (A) and cross (B) sections of a portion of a system comprising a tunnel guide and a therapy delivery element.

FIGS. 2A-2E are diagrammatic illustrations of a perspective view (A) and cross sections (B-E) of tunneling guides comprising associated therapeutic agent according to various embodiments of the invention.

FIGS. 3A and 3B are diagrammatic illustrations of cross-sections of tunneling guides showing varying concentrations of therapeutic agent disposed in and on more than one layer of tunneling guide according to embodiments of the invention.

FIGS. 4A and 4B are diagrammatic illustrations of cross-sections of tunneling guides showing varying concentrations of therapeutic agent disposed in and on layers of delivery elements according to embodiments of the invention.

FIGS. 5A and 5B are flow diagrams of methods according to embodiments of the invention.

FIGS. 6A and 6B are diagrammatic illustrations of longitudinal and cross-sections, respectively, of a tunneling guide comprising a barrier layer according to an embodiment of the invention.

The drawings are not necessarily to scale. Like numbers refer to like parts or steps throughout the drawings.

### DETAILED DESCRIPTION OF THE INVENTION

In the following description, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense. Instead, the scope of the present invention is to be defined in accordance with the appended claims.

The present disclosure describes tunneling guides capable of eluting a therapeutic agent. In various embodiments the invention provides methods, systems and devices useful for such technology. In various embodiments, the tunneling guides are configured to receive therapy delivery elements, such as leads, lead extensions, catheters, and the like. During surgery, such therapy delivery elements may be tunneled from one portion of a subject's body to another and may remain implanted in the subject for long periods of time. Accordingly, the structural integrity of therapy delivery elements is often important. Because incorporation of therapeutic elements into or onto the structural body of therapy delivery elements may compromise the structural integrity of the therapy delivery element, it is desirable to incorporate a therapeutic agent into or onto a separate tunneling guide rather than into or onto the therapy delivery device itself. Additionally, if the therapy delivery element is a catheter designed to deliver a drug, it may be desirable to prevent therapeutic agent and drug interaction by incorporating the therapeutic agent into or onto the tunneling device rather than on or in the catheter body. A tunneling guide as described herein comprises a body member and optionally one or more coating layers. The body member or the one or more coating layers comprise one or more therapeutic agents that may be eluted from the tunneling guide when the tunneling guide is contacted with bodily fluid or tissue. The eluted therapeutic agent may be useful to treat a complication associated with surgical implantation of a therapy delivery element. By incorporating the therapeutic agent on a tunneling guide, various embodiments of the invention allow for the structural integrity of a therapy delivery device to be maintained and prevent potentially undesirable interactions between the therapeutic agents and drugs that may delivered by the therapy delivery agent.

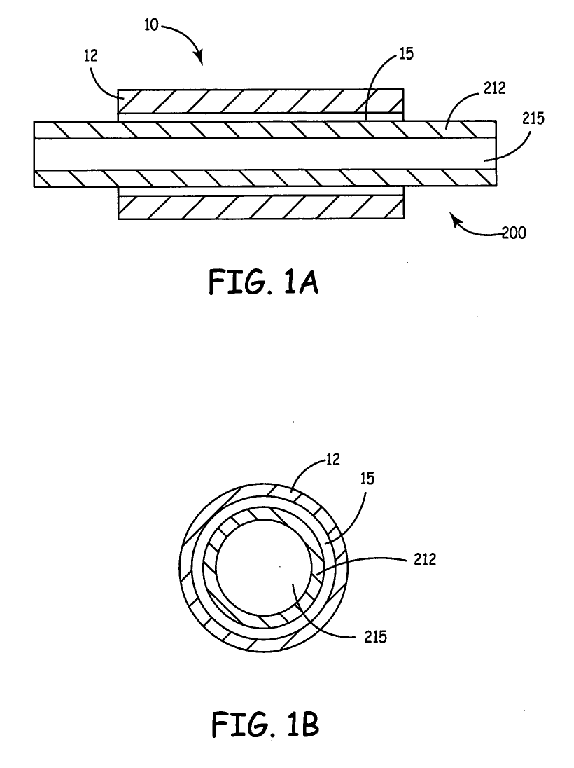

Referring to FIG. 1, an embodiment of the invention provides a system comprising a tunneling guide 10 and a therapy delivery element 200. The tunneling guide comprises a body member 12 and a lumen 15 configured to receive one or more therapy delivery elements 200. The therapy delivery element 200 comprises a structural body member 212. The therapy delivery element 200 in FIGS. 1A and 1B is a catheter comprising a lumen 215. However, it will be understood that therapy delivery element 212 may be any therapy delivery element, such as a lead, a lead extension, etc. The therapy delivery element 212 may be operably coupled to an active device (not shown), such as an infusion pump or an electrical signal generator, such as a pacemaker, a defibrillator, or a neurostimulator. The active device may be implantable.

In FIG. 1A, the tunneling guide 10 is shown to cover only a portion of the delivery element 200. However, it will be understood that tunneling guide 10 may cover all, substantially all, or any portion of the therapy delivery element 200. In an embodiment, tunneling guide is configured to be inserted into a first tissue, but not a second tissue.

The first tissue may be subcutaneous tissue. The second tissue may be CNS tissue. For example, tunneling guide 10 may be sized so that the distal end will just contact, but not penetrate, the tissues surrounding a target site; e.g. the arachnoid membrane surrounding the brain or the ligamentum flavum around the epidural space. With this configuration, tunneling guide 10 may completely fill the pathway from the device pocket to the target site, but will not enter the target site, e.g. the CNS. A therapeutic agent eluted from or associated with the tunneling guide 10 may not be toxic in subcutaneous tissue but may be neurotoxic and thus such a configuration may be desirable.

It is envisioned that an outer diameter of the delivery element 200 will generally be smaller than an inner diameter of the tunneling guide 10 to facilitate insertion of the delivery element 200 through the guide 10. However, the difference in outer diameter of the therapy delivery element 200 relative to inner diameter of the tunneling guide 10 may be minimal to effectively add mechanical strength to the delivery element 200. Thickness of body member of guide 10 may be varied to allow for ease of insertion, addition of mechanical strength, or the like.

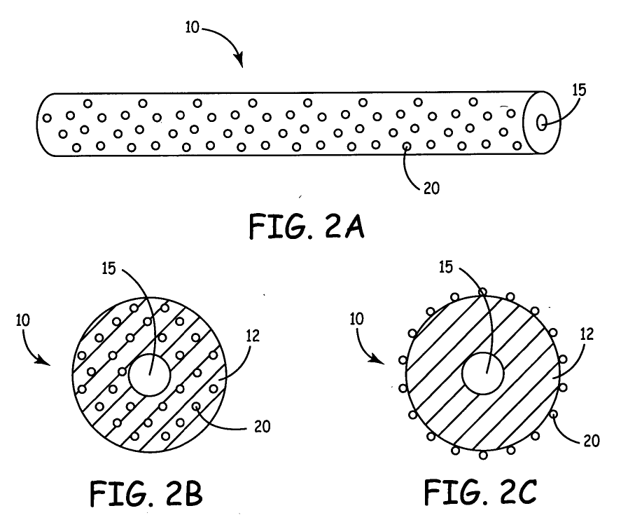

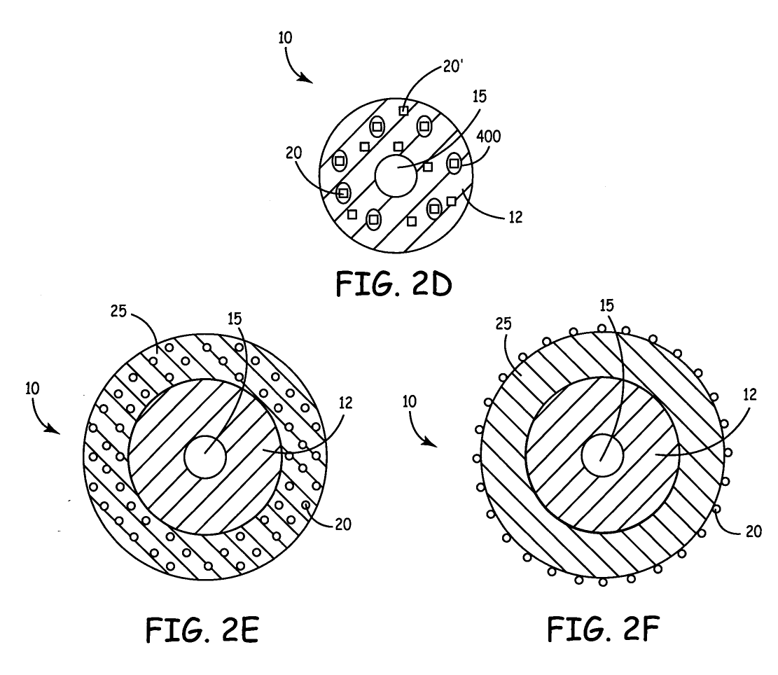

Referring to FIG. 2, an embodiment of the invention provides a therapeutic agent 20 associated with a tunneling guide 10. The tunneling guide 10 may be used to guide the implantation of at least a portion of a therapy delivery element 200 in a path through a subject. Therapeutic agent 20 may be associated with a tunneling guide 10 in any fashion such that contacting at least a portion of the tunneling guide 10 with a tissue of a subject allows for the therapeutic agent 20 to dissolve or elute into the tissue.

Any therapeutic agent 20 may be disposed in, on, or about tunneling guide 10.

Because it may be desirable to treat or prevent infections, inflammation, or proliferation associated with implantation of a medical device, it may be desirable to dispose one or more anti-infective agent, one or more anti-inflammatory agent, one or more anti-proliferative agent, or a combination thereof in, on, or about at least a portion of an external surface of tunneling guide 10. In addition, in some circumstances it may be desirable to deliver a local anesthetic. Additional or other agents 20 that may be disposed in, on, or about tunneling device 10 will be readily evident to one of skill in the art. A brief summary of some non-limiting classes of therapeutic agents that may be used follows.

1. Anti-Infective Agents

Any anti-infective agent may be used in accordance with various embodiments of the invention. As used herein, “anti-infective agent” means an agent that kills or inhibits the growth of an infective organism, such as a microbe or a population of microbes. Anti-infective agents include antibiotics and antiseptics.

A. Antibiotic

Any antibiotic suitable for use in a human may be used in accordance with various embodiments of the invention. As used herein, “antibiotic” means an antibacterial agent.

The antibacterial agent may have bateriostatic and/or bacteriocidal activities.

Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, which is herein incorporated by reference in its entirety, may also be used. One of ordinary skill in the art will recognize other antibiotics that may be used.

In general, it is desirable that the selected antibiotic(s) kill or inhibit the growth of one or more bacteria that are associated with infection following surgical implantation of a medical device. Such bacteria are recognized by those of ordinary skill in the art and include Stapholcoccus aureus, Staphlococcus epidermis, and Escherichia coli. Preferably, the antibiotic(s) selected are effective against strains of bacteria that are resistant to one or more antibiotic.

To enhance the likelihood that bacteria will be killed or inhibited, it may be desirable to combine two or more antibiotics. It may also be desirable to combine one or more antibiotic with one or more antiseptic. It will be recognized by one of ordinary skill in the art that antimicrobial agents having different mechanisms of action and/or different spectrums of action may be most effective in achieving such an effect. In an embodiment, a combination of rifampin and micocycline is used. In an embodiment, a combination of rifampin and clindamycin is used.

B. Antiseptic

Any antiseptic suitable for use in a human may be used in accordance with various embodiments of the invention. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver-containing compounds (silver sulfadiazene, silver metal, silver ion, silver nitrate, silver acetate, silver protein, silver lactate, silver picrate, silver sulfate), and alcohols. One of ordinary skill in the art will recognize other antiseptics that may be employed in accordance with this disclosure.

It is desirable that the antiseptic(s) selected kill or inhibit the growth of one or more microbe that are associated with infection following surgical implantation of a medical device. Such microbes are recognized by those of ordinary skill in the art and include Stapholcoccus aureus, Stapholococcus epidermis, Escherichia coli, Pseudomonus auruginosa, and Candidia.

To enhance the likelihood that microbes will be killed or inhibited, it may be desirable to combine two or more antiseptics. It may also be desirable to combine one or more antiseptics with one or more antibiotics. It will be recognized by one of ordinary skill in the art that antimicrobial agents having different mechanisms of action and/or different spectrums of action may be most effective in achieving such an effect. In a particular embodiment, a combination of chlorohexidine and silver sulfadiazine is used.

C. Antiviral

Any antiviral agent suitable for use in a human may be used in accordance with various embodiments of the invention. Nonlimiting examples of antiviral agents include acyclovir and acyclovir prodrugs, famcyclovir, zidovudine, didanosine, stavudine, lamivudine, zalcitabine, saquinavir, indinavir, ritonavir, n-docosanol, tromantadine and idoxuridine. One of ordinary skill in the art will recognize other antiviral agent that may be employed in accordance with this disclosure.

To enhance the likelihood that viruses will be killed or inhibited, it may be desirable to combine two or more antiviral agents. It may also be desirable to combine one or more antiseptics with one or more antiviral agent.

D. Anti-Fungal

Any anti-fungal agent suitable for use in a human may be used in accordance with various embodiments of the invention. Nonlimiting examples of anti-fungal agents include amorolfine, isoconazole, clotrimazole, econazole, miconazole, nystatin, terbinafine, bifonazole, amphotericin, griseofulvin, ketoconazole, fluconazole and flucytosine, salicylic acid, fezatione, ticlatone, tolnaftate, triacetin, zinc, pyrithione and sodium pyrithione. One of ordinary skill in the art will recognize other anti-fungal agents that may be employed in accordance with this disclosure.

To enhance the likelihood that viruses will be killed or inhibited, it may be desirable to combine two or more anti-fungal agents. It may also be desirable to combine one or more antiseptics with one or more anti-fungal agent.

2. Anti-Inflammatory Agents

Any anti-inflammatory agent suitable for use in a human may be used in accordance with various embodiments of the invention. Non-limiting examples of anti-inflammatory agents include steroids, such as cortisone, hydrocortisone, prednisone, dexamethasone, methyl-prednisilone, an derivatives thereof; and non-steroidal anti-inflammatory agents (NSAIDs). Non-limiting examples of NSAIDS include ibuprofen, flurbiprofen, ketoprofen, aclofenac, diclofenac, aloxiprin, aproxen, aspirin, diflunisal, fenoprofen, indomethacin, mefenamic acid, naproxen, phenylbutazone, piroxicam, salicylamide, salicylic acid, sulindac, desoxysulindac, tenoxicam, tramadol, ketoralac, flufenisal, salsalate, triethanolamine salicylate, aminopyrine, antipyrine, oxyphenbutazone, apazone, cintazone, flufenamic acid, clonixerl, clonixin, meclofenamic acid, flunixin, coichicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, and triflumidate.

3. Local Anesthetics

Any local anesthetic agent suitable for use in a human may be used in accordance with various embodiments of the invention. Non-limiting examples of local anesthetics agents include lidocaine, prilocaine, mepivicaine, benzocaine, bupivicaine, amethocaine, lignocaine, cocaine, cinchocaine, dibucaine, etidocaine, procaine, veratridine (selective c-fiber blocker) and articaine.

4. Other Pharmacological Agents

Non-limiting examples of other pharmacological agents that may be used include: beta-radiation emitting isotopes, beclomethasone, fluorometholone, tranilast, ketoprofen, curcumin, cyclosporin A, deoxyspergualin, FK506, sulindac, myriocin, 2-aminochromone (U-86983), colchicines, pentosan, antisense oligonucleotides, mycophenolic acid, etoposide, actinomycin D, camptothecin, carmustine, methotrexate, adriamycin, mitomycin, cis-platinum, mitosis inhibitors, vinca alkaloids, tissue growth factor inhibitors, platinum compounds, cytotoxic inhibitors, alkylating agents, antimetabolite agents, tacrolimus, azathioprine, recombinant or monoclonal antibodies to interleukins, T-cells, B-cells, and receptors, bisantrene, retinoic acid, tamoxifen, compounds containing silver, doxorubicin, azacytidine, homoharringtonine, selenium compounds, superoxide-dismutase, interferons, heparin; Antineoplastic/antiangiogenic agents, such as antimetabolite agents, alkylating agents, cytotoxic antibiotics, vinca alkaloids, mitosis inhibitors, platinum compounds, tissue growth factor inhibitors, cisplatin and etoposide; Immunosuppressant agents, such as cyclosporine A, mycophenolic acid, tacrolimus, rapamycin, rapamycin analogue (ABT-578) produced by Abbott Laboratories, azathioprine, recombinant or monoclonal antibodies to interleukins, T-cells, B-cells and/or their receptors; Anticoagulents, such as heparin and chondroiten sulfate; Platelet inhibitors such as ticlopidine; Vasodilators such as cyclandelate, isoxsuprine, papaverine, dipyrimadole, isosorbide dinitrate, phentolamine, nicotinyl alcohol, co-dergocrine, nicotinic acid, glycerl trinitrate, pentaerythritol tetranitrate and xanthinol; Thrombolytic agents, such as stretokinase, urokinase and tissue plasminogin activators; Analgesics and antipyretics, such as the opioid analgesics such as buprenorphine, dextromoramide, dextropropoxyphene, fentanyl, alfentanil, sufentanil, hydromorphone, methadone, morphine, oxycodone, papaveretum, pentazocine, pethidine, phenopefidine, codeine dihydrocodeine; acetylsalicylic acid (aspirin), paracetamol, and phenazone; and Antiproliferative agents such as QP-2 (taxol), paclitaxel, rapamycin, tacrolimus, everolimus, actinomycin, methotrexate, angiopeptin, vincristine, mitocycin, statins, C-MYC antisense, sirolimus, restenASE, 2-chloro-deoxyadenosine, PCNA (proliferating cell nuclear antigent) ribozyme, batimastat, prolyl hydroxylase inhibitors, halofuginone, C-proteinase inhibitors, and probucol; and combinations and/or derivates thereof.

Therapeutic agents 20 could include a steroid (e.g dexamethasone), a cell antiproliferative agent (e.g. rapamycin) or a radioactive substance.

Therapeutic agent 20 may be associated with tunneling guide 10 in any manner such that introduction of at least a portion of the tunneling guide 10 to a tissue of a subject allows for the therapeutic agent 20 to elute or dissolve into the tissue. FIGS. 2B-2F show examples of associations of therapeutic agent 20 with tunneling guide 10. FIG. 2B shows that therapeutic agent 20 may be disposed in a body member 12 of tunneling guide 10. While FIG. 2B shows therapeutic agent 20 disposed throughout the body member 12, the therapeutic agent 20 may be disposed within one or more portions of the body member 12 (not shown). FIG. 2C shows that therapeutic agent 20 may be disposed on the body member 12. If a therapeutic agent 20 is disposed partially within the body member 12 or other layer and partially protrudes from a surface of the body member 12 or other layer, the therapeutic agent 20 is considered both disposed in and disposed on the body member 12 or other layer. Further, while not shown, it will be understood that therapeutic agent 20 may be both disposed in and disposed on the body member 12 of the tunneling guide 10. FIG. 2D shows therapeutic agent 20 in a vector 400. The vector 400 may be disposed in, on or about body member 12. Additional therapeutic agent 20′, which may be the same or different than therapeutic agent 20, may be disposed on, in, or about body member but outside of vector 400. If therapeutic agent 20′ and therapeutic agent 20 are different and incompatible it may be desirable to incorporate therapeutic agent 20 into vector 400 to prevent premature interaction of therapeutic agents 20 and 20′, e.g., during storage of tunneling guide 10. Vector 400 may comprise a polymeric material. Vector 400 may serve as a delayed release vehicle to release therapeutic agent at the appropriate time after implantation of the tunneling guide 10. FIGS. 2E and 2F show embodiments where a coating layer 25 is disposed on the body member 12 and therapeutic agent 20 is disposed in (1D) or on (1E) the coating layer 25. As with the body member 12, therapeutic agent 20 may be disposed throughout the coating layer 25, in a portion of the coating layer 25, and/or both within and on the coating layer 25. While not shown, it will be understood that therapeutic agent 20 may be incorporated in, on, or about a vector 400, which may be incorporated in, on, or about coating layer 25.

It will be understood that therapeutic agent 20, 20′ as depicted in FIGS. 2A-2F, other subsequent Figures, and throughout the present disclosure may refer to a plurality of different therapeutic agents 20, 20′. For example, a given therapeutic agent 20, 20′ depicted in FIG. 1A may be, one or more anti-infective agents, e.g. minocycline and rifampin, one or more anti-inflammatory agents, one or more anti-proliferative agents, etc. or a combination thereof.

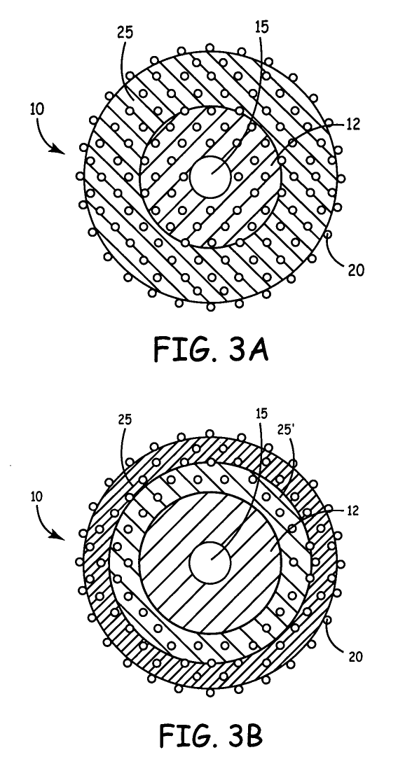

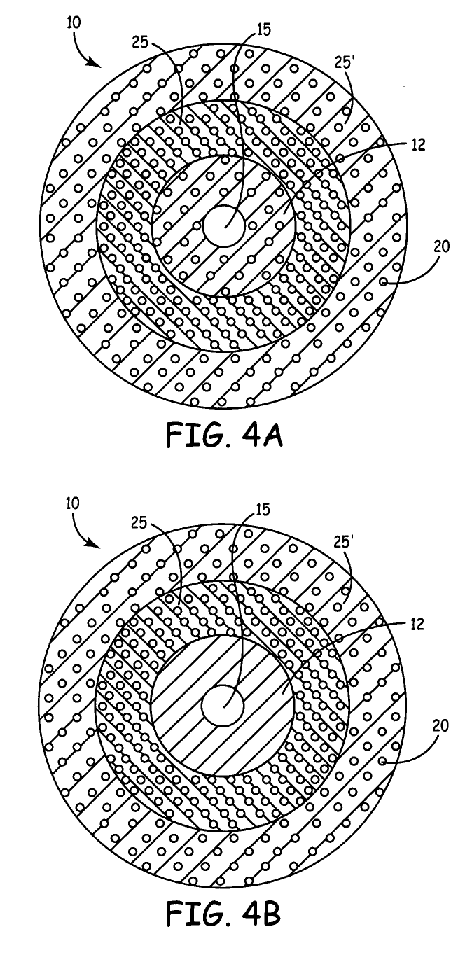

In various embodiments of the invention, therapeutic agents 20 are disposed on or in more than one layer of tunneling guide 10. For example, therapeutic agent 20 may be disposed on or in a body member 12 of tunneling guide 10, on or in one or more coating layer 25 of tunneling guide 10. FIG. 3A shows an embodiment where therapeutic agent 20 is disposed within or on body member 12 and within or on coating layer 25 of tunneling guide 10. FIG. 3B shows an embodiment where therapeutic agent 20 is disposed on or in a first coating layer 25 and on or in a second coating layer 25′. Of course, two, three, four, five, six, or more coating layers 25 may be disposed about body member 12 of tunneling guide 10 and therapeutic agent 20 may be disposed in or on the body member 12 or none, some, or all of the one or more coating layers 25.

The concentration of therapeutic agents 20 within various layers (depicted as body member 12 or coating layer 25, 25′) may be the same or different. Any concentration may be used. For example, therapeutic agent 20 may comprise about 0.1% to about 50%, or from about 1% to about 10%, of the weight of the layer. In some circumstances, it may be desirable to place a higher concentration of therapeutic agent 20 in one or more layers relative to other layers; e.g., when continued infusion of therapeutic agent 20 into body tissue over time is desired. FIG. 3A shows a tunneling guide 10, where first coating layer 25 comprises a higher concentration of therapeutic agent 20 within or on intermediate coating layer 25 than in outer coating layer 25′ or body member 12. In the embodiment illustrated by FIG. 3A, body member 12 is permeable to therapeutic agent 20 and therapeutic agent 20 may elute into lumen 15. Therapeutic agent 20 may also elute out of outer coating layer 25′ into body tissue. Increased initial concentration of therapeutic agent 20 in intermediate coating layer 25 may effectively replenish the supply of therapeutic agent 20 in outer coating layer 25′ and body member 12 such that therapeutic agent 20 may elute into lumen 15 or tissue. In the embodiment illustrated in FIG. 3B, body member 12 is essentially impermeable to therapeutic agent 20 and intermediate coating layer 25 comprises a higher concentration of therapeutic agent 20 that outer coating layer 25′. Therapeutic agent 20 in the intermediate coating layer may replenish supply in the outer coating layer 25′ over time.

Release profile of therapeutic agent 20 from tunneling guide 10, may be varied. As described above, location of therapeutic agent 20 in or on tunneling guide 10, as well as concentration of therapeutic agent 20 at a location, provides a means for achieving control over when therapeutic agent 20 is released. Release profile may be varied by controlling the nature of the therapeutic agent 20 to be released. For example, agents 20 having greater molecular mass or size may elute more slowly than agents 20 having lesser molecular mass or size. Interactions between therapeutic agent 20 and components of body member 12 or coating layer 25, 25′ may also affect the rate at which therapeutic agent is released from tunneling guide 10. With these and other considerations in mind, it may be desirable, in some circumstances, to vary the location of slower eluting therapeutic agents 20 and faster eluting therapeutic agents 20 within or on tunneling guide 10.

For example, in situations, e.g. where the tunneling guide 10 may be permanently implanted into a subject, it may be desirable to elute roughly the same amount of a therapeutic agent 20 over time. One way to achieve substantially uniform release of two or more therapeutic agents 20 over time is to dispose a slower eluting therapeutic agent 20 near surface of the tunneling guide 10 from which the agent 20 will elute and dispose a faster eluting therapeutic agent 20 further from the surface from which the agents 20 will elute. Alternatively, it may be desirable to load a substantial amount of reserve therapeutic agent 20, whether slow or fast eluting, into or on tunneling device 10, such that the reserve replenishes the supply of therapeutic agent at or near the surface of tunneling guide 10 from which the agent 20 will be released. In some situations in may be desirable to load a therapeutic agent 20 in a delayed release vector, which vector 400 is disposed in, on or about body member 12 or coating layer 25, and load different therapeutic agent 20′ in body member 12 or coating layer 25.

The rate at which therapeutic element 20 may be released from tunneling guide 10 into tissue may also be controlled by properties of coating layers 25, vector 400, or body member 12, as well as the manner in which therapeutic agent 20 is disposed on or in coating layers 25 or body member 12.

Coating Layer

Coating layer 25 may be formed of any material capable of releasing one or more therapeutic agent 20 into tissue when placed in contact with the tissue. Preferably, coating layer 25 is acceptable for at least temporary use within a human body. Coating layer 25 is also preferably compatible with therapeutic agent 20.

Examples of commonly used materials that may be used to form coating layers 25 include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE), including expanded PTFE (ePTFE).

One or more coating layer 25 according to various embodiments of the invention may comprise a biodegradable polymeric material, such as synthetic or natural bioabsorbable polymers. Synthetic bioabsorbable polymeric materials that can be used to form the coating layers include poly (L-lactic acid), polycaprolactone, poly(lactide-co-glycolide), poly(ethylene-vinyl acetate), poly(hydroxybutyrate-covalerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, and polyphosphazenes. According to another exemplary embodiment of the present invention, the polymeric materials can be natural bioabsorbable polymers such as, but not limited to, fibrin, fibrinogen, cellulose, starch, collagen, and hyaluronic acid.

Coating layers 25 may comprise polymeric materials designed to control the rate at which therapeutic agent is released, leached, or diffuses from the polymeric material. As used herein, “release”, “leach”, “diffuse”, “elute” and the like are used interchangeably when referring to a therapeutic agent 20 with respect to a coating layer 25 or body member 12 of a delivery element. Any known or developed technology may be used to control the release rate. For example, a coating layer may be designed according to the teachings of WO/04026361, entitled “Controllable Drug Releasing Gradient Coating for Medical Devices.”

Coating layer 25 of tunneling guide 10 may be in the form of a tube, sheath, sleeve, coating, or the like. Coating layer 25 may be extruded, molded, coated on body member 12, grafted onto body member 12, embedded within body member 12, adsorbed to body member 12, etc. Polymers of coating layers 25 may be porous or non-porous. Porous materials known in the art include those disclosed in U.S. Pat. No. 5,609,629 (Feamot et al.) and U.S. Pat. No. 5,591,227 (Dinh et al.). Typically polymers are non-porous. However, non-porous polymers may be made porous through known or developed techniques, such as extruding with CO₂ or by foaming the polymeric material prior to extrusion or coating.

Depending upon the type of materials used to form coating layers 25, the coatings can be applied to the surface of a body member 12 or underlying coating layer 25 through any coating processes known or developed in the art. One method includes directly bonding the coating material to a surface of body member 12 or underlying coating layer 25. By directly attaching a polymer coating to the body member 12 or underlying coating layer 25, covalent chemical bonding techniques may be utilized. Body member 12 or underlying coating layer 25 surface may possess chemical functional groups on its surface such as carbonyl groups, primary amines, hydroxyl groups, or silane groups which will form strong, chemical bonds with similar groups on polymeric coating material utilized. In the absence of such chemical forming functional group, known techniques may be utilized to activate the material's surface before coupling the biological compound. Surface activation is a process of generating, or producing, reactive chemical functional groups using chemical or physical techniques such as, but not limited to, ionization, heating, photochemical activation, oxidizing acids, sintering, physical vapor deposition, chemical vapor deposition, and etching with strong organic solvents. Alternatively, the coating layer 25 may be indirectly bound to body member 12 or underlying coating layer 25 through intermolecular attractions such as ionic or Van der Waals forces.

Therapeutic agent 20 may be incorporated into a coating layer 25 in a variety of ways. For example, therapeutic agent 20 can be covalently grafted to a polymer of the coating layer 25, either alone or with a surface graft polymer. Alternatively, therapeutic agent 20 may be coated onto the surface of the polymer either alone or intermixed with an overcoating polymer. Therapeutic agent 20 may be physically blended with a polymer of a coating layer 25 as in a solid-solid solution. Therapeutic agent 20 may be impregnated into a polymer by swelling the polymer in a solution of the appropriate solvent. Any means of incorporating therapeutic agent 20 into or on a coating layer 25 may be used, provided that therapeutic agent 20 may be released, leached or diffuse from coating layer 25 on contact with bodily fluid or tissue.

A polymer of a coating layer 25 and a therapeutic agent 20 may be intimately mixed either by blending or using a solvent in which they are both soluble. This mixture can then be formed into the desired shape or coated onto an underlying structure of the medical device. One exemplary method includes adding one or more therapeutic agents 20 to a solvated polymer to form a therapeutic agent 20/polymer solution. The therapeutic agent 20/polymer solution can then be applied directly to the surface of body member 12 or underlying coating layer 25; for example, by either spraying or dip coating tunneling guide 10. As the solvent dries or evaporates, the therapeutic agent 20/polymer coating is deposited on delivery element 12. Furthermore, multiple applications can be used to ensure that the coating is generally uniform and a sufficient amount of therapeutic agent 20 has been applied to tunneling guide 10.

Alternatively, an overcoating polymer, which may or may not be the same polymer that forms the primary polymer of body member 12 or underling coating layer 25, and therapeutic agent 20 are intimately mixed, either by blending or using a solvent in which they are both soluble, and coated onto body member 12 or underling coating layer 25. Any overcoating polymer may be used, as long as the polymer is able to bond (either chemically or physically) to the polymer of an underlying layer of tunneling guide 10.

In addition, a polymer of a coating layer 25 may be swelled with an appropriate solvent, allowing a therapeutic agent 20 to impregnate the polymer.

Therapeutic agent 20 may also be covalently grafted onto a polymer of a coating layer 25.

This can be done with or without a surface graft polymer. Surface grafting can be initiated by corona discharge, UV irradiation, and ionizing radiation. Alternatively, the ceric ion method, previously disclosed in U.S. Pat. No. 5,229,172 (Cahalan et al.), may be used to initiate surface grafting.

Body Member

Body member 12 of tunneling guide 10 may be made of any material onto or into which a therapeutic agent 20 may be disposed or onto or into which a coating layer 25, 25′ comprising a therapeutic agent 20 may be directly or indirectly disposed, such that the therapeutic agent 20 may be released into tissue or bodily fluid when tunneling guide 10 is contacted with the tissue or fluid. Preferably, body member 12 is formed of material acceptable for at least temporary use within a human body. Preferably body member 12 is made of material of sufficient rigidity such that lumen 15 serves as a suitable conduit for therapy delivery element 200 and is of sufficient flexibility to tunnel a path within a subject, which path may be non-linear. In some embodiments, a body member 12 made of a metallic material may have sufficient properties for its intended purpose. In other embodiments, body member 12 may be formed of a polymeric material. Suitable polymeric materials include those described above for coating layers 25, 25′. In various embodiments, body member 12 is made of silicone, polyurethane, or expanded polytetraflourethylene (ePTFE).

Body member 12 may comprise a metallic material. A metallic body member 12 may be porous or sintered. A body member 12 comprising metallic material may be treated by, e.g., ionization, heating, photochemical activation, oxidizing acids, sintering, physical vapor deposition, chemical vapor deposition and/or etching with strong organic solvents, as discussed above, to facilitate disposing therapeutic agent 20 directly on the body member 12. One or more coating layers 25, 25′, one or more of which may comprise a therapeutic agent 20, may be disposed on body member according to the teachings described herein. Body member 12 may optionally be treated as discussed above to facilitate coating of a polymeric material onto the body member 12.

A coating layer 25 in the form of a sheath, sleeve, or the like may be disposed about a needle electrode 30. The sheath, sleeve, etc. may be disposable or designed for one use.

Vector

Vector 400 may be formed of any material capable of releasing one or more therapeutic agent 20 into body member 12, coating layer 25 or tissue when tunneling guide 10 is placed in contact with the tissue. Generally, vector 400 may comprise material suitable for forming a coating layer 25.

Vector 400 may be disposed within, on, or about coating layer 25 or body member 12 in a manner substantially similar to disposing therapeutic agent 20 in, on, or about coating layer 25 or body member 12. For example, vector 400 may be compounded into a polymeric material of body member 12 or coating layer 25 and extruded with body member 12 or coating layer, or vector 400 may be mixed in fluid form with a polymeric material of coating layer 25, which mixture may then be applied to body member 12. These and other techniques, whether known or future developed, may be used to dispose vector 400 in, on, or about body member 12 or coating layer 25.

Therapeutic agent 20 may be incorporated in, on, or about vector 400 as described herein for disposing therapeutic agent 20 in, on or about coating layer 25.

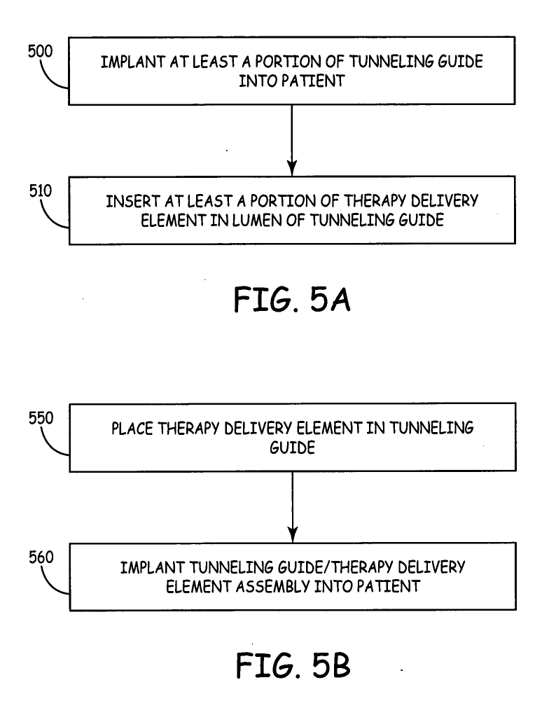

Systems and apparatuses described herein may be implanted in a patient in any medically acceptable manner. One exemplary method is shown in FIG. 5A. At 510, tunneling guide 10 is implanted into a patient, and at 520 at least a portion of therapy delivery element 200 is inserted into lumen 15 of tunneling guide 10. It will be understood that inserting at least a portion of therapy delivery element into lumen of tunneling guide 10 may comprise: inserting one or more additional apparatuses (not shown), such as a barrier layer tube discussed below, having a lumen into the lumen 15 of tunnel guide; and inserting therapy delivery element 200 into the lumen of at least one of the one or more additional apparatuses. Lubricants may be employed to facilitate placement of therapy delivery element in lumen 15 of guide 10. For example, lubricants may be placed in lumen 15 of tunneling guide 10 or on therapy delivery element 200. A lubricant can be used to reduce friction between the catheter or lead and the tunnel guide. Any lubricant may be used. Non-limiting examples of suitable lubricants include saline, cod liver oil, or mineral oil. Alternatively, either the outer surface of the catheter or lead, or the inner surface of the tunnel guide can be coated with a lubricious hydrogel, or similar lubricious material. Other suitable methods may also be used to implant systems and apparatuses described herein. For example, therapy delivery element 200 may be implanted in a patient, and guide 10 may be placed about at least a portion of delivery element 200, or as shown in FIG. 5B therapy delivery element 200 may be placed in guide 10 (550) prior to implantation and the assembly may then be implanted into a patient (560).

Barrier Layer

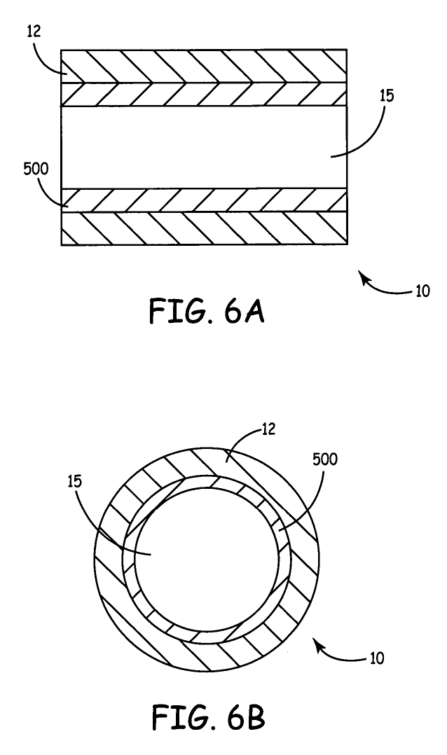

In various embodiments, a diffusion barrier 500 may be disposed on inner surface of body member 12, such that diffusion barrier 500 defines at least a portion of the lumen 15 of the tunneling guide 10 (see FIGS. 6A and B). Diffusion barrier 500 may be formed of any material capable of reducing or eliminating diffusion of a therapeutic agent 20, 20′ from body member 12 to lumen 15. Diffusion barrier 500 may be a material as described above for body member 15 or coating layer 25, 25′. One suitable material for forming diffusion barrier 500 is polytetrafluoroethylene (PTFE). Another suitable material is a layer of metal, such as titanium. Diffusion barrier 500 may be disposed on body member 12 as described herein for coating layer 25, 25′.

While not shown, it will be understood that diffusion barrier 500 may be separate from body member 12. For example, diffusion barrier 500 may be a separate tube disposed between at least a portion of tunneling guide 10 and therapy delivery element 200, or diffusion barrier 500 may be disposed on therapy delivery element 200. Diffusion barrier 500 may serve to reduce friction between therapy delivery element 200 and tunneling guide.

All printed publications, such as patents, patent applications, technical papers, and brochures, cited herein are hereby incorporated by reference herein, each in its respective entirety. As those of ordinary skill in the art will readily appreciate upon reading the description herein, at least some of the devices and methods disclosed in the patents and publications cited herein may be modified advantageously in accordance with the teachings of the present invention.

What is claimed is:

1. An implantable tunneling guide comprising:
a body member defining a lumen, the lumen configured to receive at least a portion of a therapy delivery element; and
one or more therapeutic agents disposed on, in, or about the body member.

2. The implantable tunneling guide of claim 1, wherein the lumen is configured to receive at least a portion of a lead, a lead extension, or a catheter.

3. The tunneling guide of claim 1, wherein the lumen comprises a diffusion barrier to prevent at least a percentage of at least one of the one or more therapeutic agents from diffusing through the lumen.

4. The tunneling guide of claim 3, wherein the diffusion barrier comprises polytetrafluoroethylene (PTFE) or a metal layer.

5. The tunneling guide of claim 4, wherein the metal layer comprises titanium.

6. The implantable tunneling guide of claim 1, wherein the body member comprises a polymeric material.

7. The implantable tunneling guide of claim 6, wherein the body member comprises silicone or polyurethane.

8. The implantable tunneling guide of claim 1, wherein the body member comprises a coating layer and the one or more therapeutic agents are disposed on, in, or about the coating layer.

9. The implantable tunneling guide of claim 1, wherein the one or more therapeutic agents are selected from the group consisting of anti-infective agents, anti-inflammatory agents, and local anesthetics.

10. The implantable tunneling guide of claim 1, wherein the one or more therapeutic agents comprise an antibiotic.

11. The implantable tunneling guide of claim 1, wherein the one or more therapeutic agent comprises an anti-infective agent selected from the group consisting of minocycline, rifampin, clindamycin, and silver sulfadiazene.

12. The implantable tunneling guide of claim 1, wherein the one or more therapeutic agents comprise minocycline and rifampin.

13. The implantable tunneling guide of claim 1, wherein the guide is configured to be inserted into a first tissue but not into a second tissue.

14. The implantable tunneling guide of claim 13, wherein the first tissue is subcutantous tissue.

15. The implantable tunneling guide of claim 13, wherein the second tissue is central nervous system tissue.

16. A system comprising:
a therapy delivery element; and
an implantable tunneling guide comprising a body member defining a lumen and one or more therapeutic agents disposed on, in, or about the body member, the lumen configured to receive at least a portion of the therapy delivery element.

17. The system of claim 16, wherein the therapy delivery element is selected from the group consisting of a lead, a lead extension, or a catheter.

18. The system of claim 16, wherein the body member comprises a polymeric material.

19. The system of claim 16, wherein the body member comprises a coating layer and the one or more therapeutic agents are disposed on, in, or about the coating layer.

20. The system of claim 16, wherein the one or more therapeutic agents are selected from the group consisting of anti-infective agents, anti-inflammatory agents, and local anesthetics.

21. The system of claim 16, wherein the one or more therapeutic agents comprise an antibiotic.

22. The system of claim 16, wherein the one or more therapeutic agent comprises an anti-infective agent selected from the group consisting of minocycline, rifampin, clindamycin, and silver sulfadiazene.

23. The system of claim 16, wherein the one or more therapeutic agents comprise minocycline and rifampin.

24. The system of claim 16 further comprising a barrier layer disposed between the body member of the tunneling guide and the therapy delivery element.

25. The system of claim 24, wherein the lumen of the tunneling guide comprises the barrier layer.

26. The system of claim 24, wherein the barrier layer comprises a tube separate from the tunneling guide and the therapy delivery element.

27. The system of claim 24, wherein the barrier layer is disposed on the therapy delivery element.

28. A method for delivering a therapeutic agent associated with implantation of a therapy delivery element, the method comprising:
implanting a tunneling guide within a patient, the tunneling guide comprising a lumen defined by a body member and comprising one or more therapeutic agents disposed on, in, or about the body member;
inserting at least a portion of the therapy delivery element into the lumen of the tunneling guide.

29. The method of claim 28, wherein the inserting at least a portion of the therapy delivery element comprises inserting at least a portion of a catheter, a lead, or a lead extension.

30. The method of claim 28, further comprising lubricating the lumen to facilitate insertion of the at least a portion of the therapy delivery element into the lumen.

31. The method of claim 28, further comprising disposing a barrier layer between the body member of the tunneling guide and the therapy delivery element.

32. The method of claim 28, wherein the therapy delivery element is inserted into the lumen of the tunneling guide prior to implanting a tunneling guide within a patient.
